(19) United States
(12) Patent Application Publication
(10) Pub. No.: US 2022/0111085 A1
(43) Pub. Date: Apr. 14, 2022

(54) MULTIFUNCTION UV DISINFECTOR

(71) Applicant: Chenghung Pan, Palo Alto, CA (US)

(72) Inventor: Chenghung Pan, Palo Alto, CA (US)

(21) Appl. No.: 17/138,332

(22) Filed: Dec. 30, 2020

Related U.S. Application Data
(60) Provisional application No. 63/091,324, filed on Oct. 14, 2020.
(60) Provisional application No. 63/109,346, filed on Nov. 4, 2020.
(60) Provisional application No. 63/115,615, filed on Nov. 19, 2020.

(51) Int. Cl. A61L 2/10; A61L 2/24; A61L 2/26; A61L 9/20

(52) U.S. Cl. CPC A61L 2/10; A61L 2/24; A61L 2/26; A61L 9/20; A61L 2202/11; A61L 2209/14; A61L 2202/15; A61L 2202/16; A61L 2209/12; A61L 2209/134; A61L 2202/14

(57) ABSTRACT

A multifunction UV disinfector includes a blower, a UV source, and a housing with a shield mechanism. The shield mechanism provides a closed configuration that directs an air flow created by the blower past the UV source for air disinfection and that shields a surroundings of the disinfector from the UV source. The shield mechanism further provides an open configuration in which the UV light from the UV source is directed out of the disinfector to surfaces for surface disinfection in the surroundings. The disinfector may further employ a filter or an ionizer.

### CROSS-REFERENCE TO RELATED APPLICATIONS

This patent document claims benefit of the earlier filing dates of U.S. provisional Pat. App. No. 63/091,324, entitled “An Ultraviolet Disinfector with Air Filters and Removable Covers,” filed Oct. 14, 2020, U.S. provisional Pat. App. No. 63/109,346, entitled “A 2-in-1 UVC Disinfector with both Air and Surface Disinfection Capability,” filed Nov. 4, 2020, and U.S. provisional Pat. App. No. 63/115,615, entitled “A 4-in-1 UVC Disinfection with UVC Air Disinfection, HEPA filters, Needlepoint Bipolar Ionizer and UVC Surface Disinfection Functions,” filed Nov. 19, 2020, which are hereby incorporated by reference in their entirety.

### BACKGROUND

Ultraviolet (UV) light is electromagnetic radiation with wavelengths shorter than visible light but longer than X-rays. UV light is generally categorized into several wavelength ranges, and short-wavelength UV light having wavelengths between about 200 nm and 300 nm, sometimes referred to as UVC light, is considered to be “germicidal UV.” In particular, organic materials such as nucleic acids strongly absorb UV wavelengths between about 200 nm and 300 nm, and the energy that an organic organism such as bacteria or viruses absorbs can result in the death or inactivation of the organism. UV light has accordingly been used for disinfection purposes, for example, to inactivate harmful microbes.

Surface UV disinfectors are typically used to disinfect surfaces in a room or other environment. In particular, a surface UV disinfector may irradiate floors, wall, or the surfaces of furnishings within a room with a sufficient dose of UV radiation to kill or inactivate target microbes on the surfaces. Surface disinfectors are generally used in an environment that is unoccupied, e.g., at night, because safely shielding occupants from the emitted UV radiation during surface disinfection may be difficult or impractical.

An air disinfector or air purifier can trap contaminants or contagions from the ambient air and is usually used in a room or other environment while the environment is occupied, e.g., during the day, working, or business hours when occupants seek to avoid airborne contaminants or contagions. A conventional air disinfector may, for example, include a fan or blower that draws air from an environment, directs the air through a High-Efficiency Particulate Absorbing (HEPA) filter that removes contagions from the air, and returns filtered air to the environment. An issue for many conventional air disinfectors is the need to service a HEPA filter that may contain accumulated contagions.

Current disinfections systems general required separate equipment for surface disinfection and air disinfection. Obtaining, operating, and maintaining multiple disinfection systems can be inconvenient and expensive.

### SUMMARY

In accordance with an aspect of the present disclosure, a multifunction UV disinfector may function as an air disinfector during the day or when an environment is occupied and also function as a UV surface disinfector at night or when the environment is unoccupied. Since an air disinfector and a surface UV disinfector are used at different times, e.g., during time periods that do not overlap, one piece of equipment can serve multiple purposes.

In accordance with another aspect of the current disclosure, a UV air disinfector can employ UV radiation to kill or inactivate contagions that might otherwise contaminate an HEPA filter. A multifunction disinfector may particularly include one or more of a UVC air disinfector, a HEPA or other mechanical filter, a carbon or other chemical filter, and an ionizer that may be used when disinfecting or filtering air. The multifunction UV disinfector further includes a shield for a UVC source used in the UVC air disinfector, and the shield may have a closed state and an open state. The shield in the closed state protects occupants of an environment from UV radiation, e.g., during air disinfection, and the shield in the open state allows UV radiation to reach surfaces in an environment being disinfected, e.g., during surface disinfection when the environment is unoccupied.

### BRIEF DESCRIPTION OF THE DRAWINGS

FIG. 1 shows a block diagram of a multifunction UV disinfector in accordance with an example of the present disclosure.

FIGS. 2-1, 2-2, and 2-3 respectively show an example of the present disclosure with a perspective view of a multifunction UV disinfector having removable panels in place for air disinfection, a cross-section of one of the removable panels of the UV disinfector, and a perspective view showing the multifunction UV disinfector with the panels removed for surface disinfection.

FIGS. 3-1 and 3-2 respectively show a multifunction UV disinfector in accordance with an example of the present disclosure having a removable cylindrical cover in position for air disinfection and removed for surface disinfection.

FIG. 4 shows a multifunction UV disinfector in accordance with an example of the present disclosure having a retractable cylindrical cover in a raised position for surface disinfection.

FIGS. 5-1 and 5-2 show a multifunction UV disinfector in accordance with an example of the present disclosure having a retractable UV light source respectively in a lowered or closed position for air disinfection and in a raised or open position for surface disinfection.

FIGS. 6-1 and 6-2 show a base of a multifunction UV disinfector in accordance with an example of the present disclosure.

FIG. 7 is a flow diagram of a process for operating a multifunction UV disinfector in accordance with an example of the present disclosure.

The drawings illustrate examples for the purpose of explanation and are not of the invention itself. Use of the same reference symbols in different figures indicates similar or identical items.

### DETAILED DESCRIPTION

In accordance with an aspect of the current disclosure, a multifunction UV disinfector may be used for both air disinfection or surface disinfection. Air disinfection is commonly employed in an occupied environment to protect occupants from airborne contagions, and UV surface disinfection is commonly employed to remove contagions on surfaces while the environment is unoccupied and therefore people are not subjected to UV radiation. In general, both types of disinfection are desirable in the same environments, e.g., workplaces and commercial establishments, but the different types of disinfection are used at different times, e.g., air disinfection during working or business hours, and surface disinfection when a business is closed. Accordingly, a multifunction UV disinfector can serve both functions at different times, minimizing costs and optimizing equipment utilization.

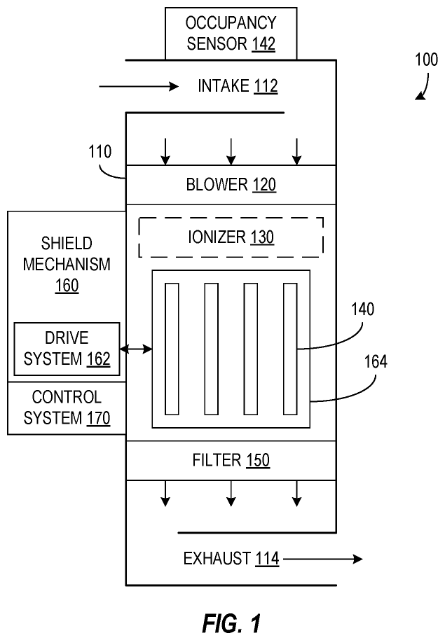

FIG. 1 is a block diagram of a multifunction UV disinfector 100 in accordance with an example of the current disclosure. Disinfector 100 includes a housing 110. Housing 110 contains major functional components including a blower 120, an ionizer 130, a UV source 140, a mechanical or chemical filter 150 of multifunction disinfector 100. Housing 110 in a closed configuration acts as a shield that keeps radiation from UV source 140 within an air flow path of disinfector 100 when UV disinfector 100 is operating only as an air disinfector. In the illustrated configuration of FIG. 1, housing 110 defines an intake vent 112 to receive or draw in air from an environment containing disinfector 100 and an exhaust vent 114 out of which disinfected air returns to the environment. Housing 110 further has a shield mechanism 160 that maintains UV shielding around UV source 140 while housing 110 is in the closed configuration. Shield mechanism 160 also opens to release or direct UV radiation from UV source 140 into the environment for surface disinfection, e.g., when the environment is unoccupied and people and animals do not need or require protection from UV. As described further below, shield mechanism 160 may include panels, covers, mountings, or other passive elements that form parts of housing 110 and further include active elements such a motor or drive system 162 capable or powered operation to move one or more components of disinfector 100, e.g., a UV source 140 or a cover 164, between the closed configuration and the open configuration.

Blower 120 may be an electrical fan or other air moving mechanism that drives an air flow through disinfector 100. Blower 120 in the illustrated configuration particularly draws air from the surrounding environment into intake 112, through the air flow path of disinfector, e.g., through ionizer 130, around UV source 140, through filter 150, and out of exhaust vent 114 back to the surrounding environment.

Ionizer 130 may serve multiple purposes. Ionizer 130 may, for example, be a needlepoint bipolar ionizer that improves filtering or produces beneficial ions. Alternatively, or additionally, ionizer 130 may include any type of ionizing filter that charges and electrically removes particles from air.

UV source 140 may include a discharge lamp or LED lamp that produces germicidal ultraviolet radiation or UVC at intensity levels capable of killing or inactivating target microbes on surfaces at distances expected in an environment to be disinfected. For example, a UVC source having an intensity of about 0.6 mW/cm² may be able to inactivate many types of microbes at distances of up to about 10 m in an exposure time of about 10 minutes. More generally, higher or lower power UVC sources may be employed depending on the distance to a surface being disinfected and the maximum exposure times considered acceptable. As noted above, when housing 110 is in the closed configuration, UV radiation from UV source 140 is kept within housing 110 and can kill or inactivate microbes passing by UV source 140, e.g., microbes in the air flow through disinfector 100. The surface disinfection function of multifunction UV disinfector 100 is employed when shield mechanism 160 of housing 110 is in the open configuration. An occupancy sensor 142, e.g., a motion sensor, may be used as part of a safety system that shuts off UV source 140 when occupants are detected around UV disinfector 100 while shield mechanism 160 is in the open configuration.

UV source 140 may be positioned to irradiate air flow or surfaces outside UV disinfector 100 and also to irradiate ionizer 130 and filter 150. As a result, UV source 140 can inactivate microbes that may be collected or trapped by ionizer 130 or filter 150 on surfaces within disinfector 100. This disinfection of ionizer 130, filter 150, and other portions of disinfector 100 make disinfector 100 less hazardous to maintain. In particular, filter 150 (or ionizer 130) may require periodic removal and servicing or replacement when a quantity of contaminates have been captured. Removal or servicing may be safer because UV source 140 inactivates contagions that filter 150 or ionizer 130 may have captured.

Filter 150 may be a mechanical or chemical filter. In one specific configuration of disinfector 100, filter 150 is a HEPA filter. HEPA refers to efficiency standards for air filters and is an acronym of high-efficiency particulate air, high-efficiency particulate absorbing, or high-efficiency particulate arrestance. Filters meeting a HEPA standard must satisfy corresponding levels of efficiency. Common standards require that a HEPA air filter removes at least 99.95% (European Standard) or 99.97% (ASME, U.S. DOE) of particles with diameter of 0.3 μm from the air that passes through the filter. A HEPA standard may required different filtration efficiency for particles of other sizes. More generally, filter 150 traps or captures particles from an air flow through filter 150 and over time may become clogged. In addition to trapping particles, filter 150 may include a chemical agent, e.g., carbon, that is effective at absorbing particular chemicals from the air flow. Mechanical and chemical filters typically require maintenance, e.g., replacement or cleaning, when the filter becomes less effective.

A process for using multifunction disinfector 100 may include placing disinfector 100 in position to draw air from an environment during times that the environment is occupied by people or animals that may benefit from air disinfection. Disinfector 100 is then in the closed configuration to protect the occupants from UV radiation, while blower 120 and UV source 140 (as well as one or more of ionizer 130 and filter 150) are activated to cause an air flow circulation to and from the environment that disinfects the air and may remove contagions, particles, and chemicals from the air and inactivate contagions that may or may not be trapped in disinfector 100. The process may further include opening shield mechanism 160 of housing 110 while disinfector 100 is in position so that UV radiation 140 is directed out of shield mechanism 160 of housing 110 to disinfect one or more surfaces in the environment. The surface disinfection may be conducted while the environment is not occupied by people or animals that the UV radiation may harm. The surface disinfection may include opening shield mechanism 160 while disinfector 100 is positioned for air disinfection, e.g., if disinfector 100 has a fixed mounting location, or moving disinfector 100 to one or more locations in the environment for surface disinfection controlled using a timer or other control system.

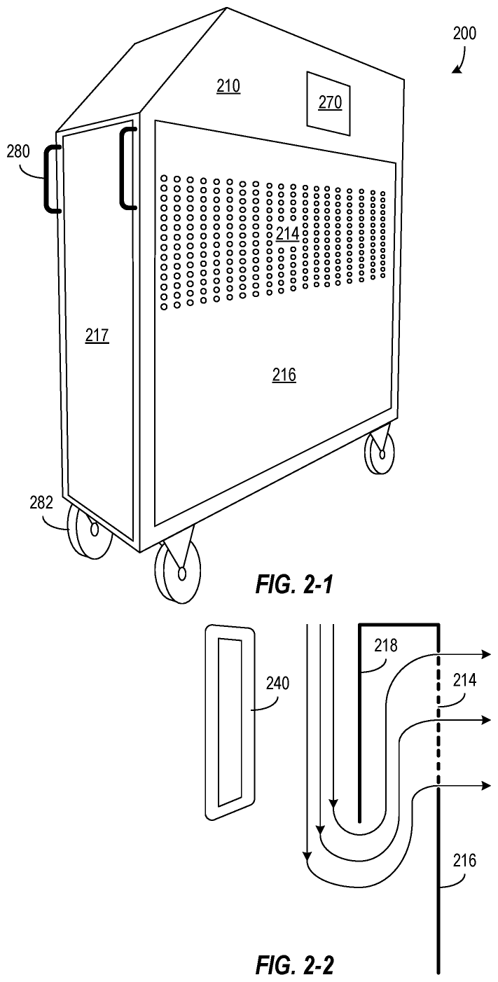

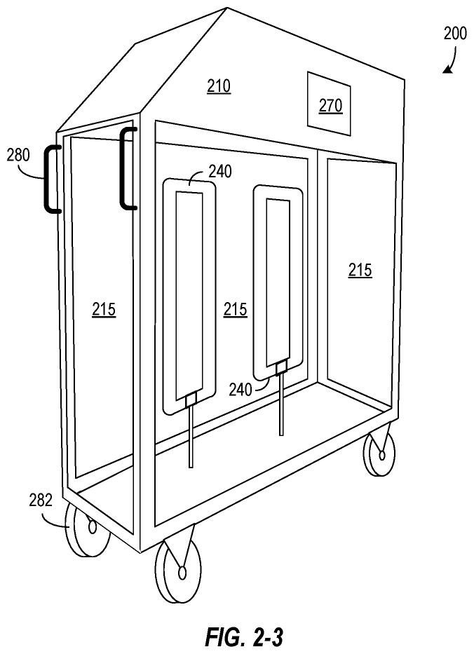

FIG. 2-1 shows one example of a multifunction UV disinfector 200 configured for air disinfection. Disinfector 200 particularly includes a housing 210 with a shield mechanism in the closed configuration. Housing 210 is generally box shaped and has multiple removable panels 216 and 217 that together form the shield mechanism. In different examples, housing 210 includes an intake vent and an exhaust vent, either of which may be located on the top, bottom, or a side, e.g., a panel 216 or 217, of housing 210. FIG. 2-2 shows an example in which removable panel 216 includes a vent 214. Vent 214 may be an intake or exhaust vent, but for purposes of illustration, vent 214 is presumed to be an exhaust vent. Panel 216 further includes a baffle or shield portion 218 that is positioned between vent 214 and UV sources 240 of disinfector 200 when panel 216 is installed in housing 210, e.g., when the shield mechanism is closed.

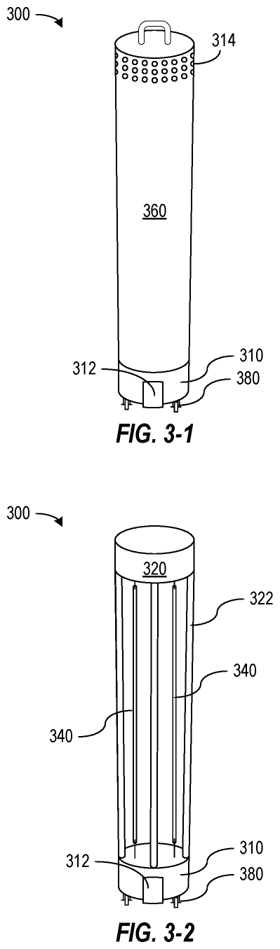

FIG. 2-3 shows multifunction UV disinfector 200 when all of the panels 216 and 217 of the shield mechanism are removed to configure disinfector 200 for surface disinfection. In particular, openings 215 through each wall of housing 210 expose UV sources 240 so that UV sources 240 can irradiate surfaces in the environment around disinfector 200. Disinfector 200 also includes handles 280 and wheels or casters 282 that facilitate moving disinfector 200 around its environment to more effectively irradiate surfaces at any locations in the environment. A control system 270 of disinfector 200 may include an occupation sensor, e.g., a motion sensor, that senses whether the environment is occupied and a controller shuts or keeps UV sources 240 off when the environment is occupied. Control system 270 may further include timers and provides a user interface that allows a user to activate surface disinfection according to a schedule, e.g., to switch UV disinfector 200 between air disinfection and surface disinfection or to start and stop air or surface disinfection at specified times or to start operations at specified times for user-specified durations. Control system 270 may also provide for control of air disinfection, e.g., to control timing of air disinfection or control air flows, e.g., fan speeds. Control system 270 may further include a Wi-Fi interface or other wireless or wired network or communication interface that permits control of UV disinfector 200 from a remote location, e.g., through a local network or through a wide area network or public network such as the Internet.

FIG. 3-1 shows a multifunction UV disinfector 300 configured for air disinfection. Disinfector 300 particularly has a housing that includes a base 310 and a removable cover 360. Base 310 includes an air vent 312 and cover 360 includes an air vent 314. In different examples of UV disinfector 300, vent 312 or 314 may be an intake vent and the other vent 314 or 312 may be an exhaust vent. In the example of FIGS. 3-1 and 3-2, cover 360 is a removable cylinder with a top and handle that forms a shield mechanism used when switching UV disinfector from air disinfection to surface disinfection. FIG. 3-1 shows disinfector 300 in the closed configuration with cover 360 in place and covering UV sources 340 of disinfector 300 and therefore shielding the surrounding environment from UV radiation during UV air disinfection.

FIG. 3-2 shows UV disinfector 300 in the open configuration with cover 300 removed to expose UV sources 340 for surface disinfection processes. In the open configuration, UV sources may be activated to direct UV light into the surrounding environment to irradiate and disinfect surfaces in the environment. Wheels or casters 380 on base 310 allow UV disinfector 300 to be move as need to different locations to effectively disinfect all desired surfaces in the environment. The open configuration of FIG. 3-2 also illustrates that multifunction disinfector 300 may have functional components in base 310 below UV sources 340, and in a top section 320 above UV sources 340 on supports 322. In one example implementation, top section 320 includes a blower that during air disinfection draws air from the surrounding environment through vent 314, directs the air down along UV sources 340 for UV disinfection, before the air heads out through a HEPA filter and vent 312 in base 310.

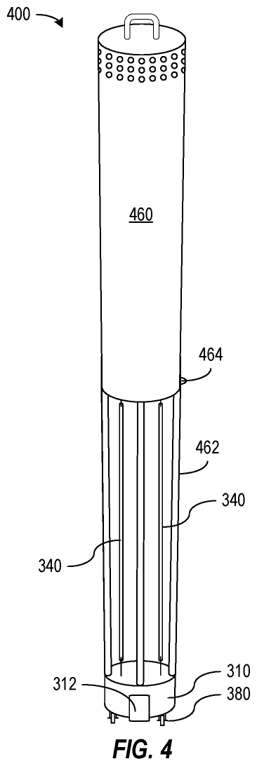

Instead of having a removable cover or shield, a shield mechanism for a multifunction UV disinfector may have a cover or shield that swings or slides open but remains attached to the UV disinfector. FIG. 4 shows an example of a multifunction UV disinfector 400 that may be substantially identical to multifunction UV disinfector 300, except that UV disinfector 400 has a shield or cover 460, which replaces cover 360 of UV disinfector 300. Cover 460 may have a hollow cylindrical shape with a sliding mount including internal guides that slide up on support rails 462 extending between base 310 and top section 320. When cover 460 is in the closed configuration for air filtration, multifunction UV disinfector 400 may look and operate in the same manner as UV disinfector 300 shown in FIG. 3-1. For surface disinfection, cover 460 is slid up as shown in FIG. 4 to expose UV light sources 340. One or more latch mechanisms 464, e.g., a spring loaded stop button that engage a hole through cover 460, on support rails 462 or other upper portion of UV disinfector 400 can hold cover 460 in the open configuration during surface disinfection. Latch mechanisms 464 may be released to lower cover 460 by to the closed configuration. Alternatively, a motorized drive system may be employed to raise or lower cover 460 on support rails 462. An advantage of a sliding cover or shield such as cover 460 that remains attached to a UV disinfector such as UV disinfector 400 is that the cover or shield does not need to be stored when not in use, which avoids the inconvenience of providing a storage location and avoids the chance of the cover being misplaced.

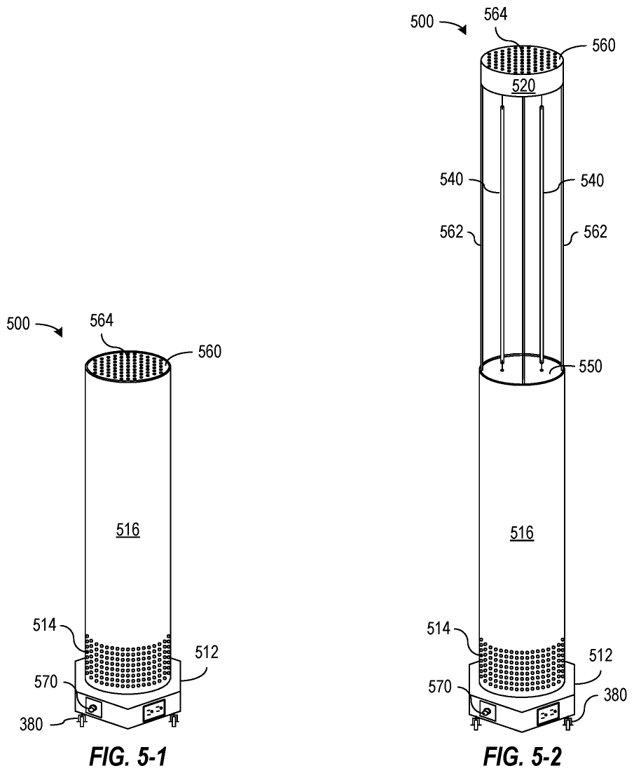

An alternative to a shield mechanism with movable or removable covers or shields is a shield mechanism with a movable UV source. For example, a shield mechanism may include a sliding mount for a UV source that can be moved into a shielding structure to reach a closed or shielded configuration for air disinfection and moved out of the shielding structure to reach an open or unshielded configuration for surface disinfection. FIGS. 5-1 and 5-2, for example, show a multifunction UV disinfector 500 respectively in a closed configuration and an open configuration. Multifunction disinfector 500 has a housing including a base 512 and a shield 516 attached to base 512. A lower vent 514 is integrated into base 512 or shield 516. In the illustrated configuration, base 512 is hexagonal and shield 516 is cylindrical but other shapes could be used. Shield 516 is hollow and contains a sliding insert 560, e.g., an insert on a sliding mount and connected to a motor or other drive system. Sliding insert 560 provides a framework 562 on which functional elements, e.g., a blower 520, UV light sources 540, and a filter 550, or UV disinfector 500 may be mounted as shown in FIG. 5-2. Sliding insert 560 also include a upper vent 564 of multifunction UV disinfector 500.

In the closed configuration shown in FIG. 5-1, insert 550 is surrounded by shield 516, and vent 564 is located at or near the top of UV disinfector 500. For air disinfection, blower 520 can be activated to push an air flow, e.g., into vent 564, down through shield 516, through filter 550, and out of vent 514. During the air disinfection, UV light sources 540 may be on so that UV light can inactivate contagions in the air flow and that may be trapped in filter 550 or elsewhere inside UV disinfector 500. Shield 516, filter 550, and blower 520 enclose UV light sources 540 so that UV light from UV sources 540 cannot escape from UV disinfector 500 in the closed configuration of FIG. 5-1.

In the open configuration shown in FIG. 5-2, insert 560 is slid up and latched in a position where UV sources 540 are exposed and able to emit UV light for surface disinfection. UV sources 540 may be activated to emit UV light for surface disinfection only when an occupancy detector determines that the surrounding around UV disinfector 500 is unoccupied. An advantage of using an insert 560 able to move UV sources 540 up for surface disinfection is that even if UV disinfector 500 has a low profile, e.g., around 4 feet tall or less, in the closed configuration, the open configuration is taller, e.g., about 6 feet tall, and positions UV light source 540 higher for surface disinfection. Multifunction UV disinfector 500 can thus provide a low and less conspicuous profile for air disinfection but still position UV sources 540 at a height well suited for surface disinfection at height typical of human activity in a room or other environment.

Multifunction UV disinfector 500 further includes a control system 570 that enables a user interface for control of UV disinfector 500. Control system 570 may, for example, include a manual button or switch that a user can depress or operate to change UV disinfector 500 between air disinfection (closed state) and surface disinfection (open state) operation. In particular, pressing a button may cause control system 570 to activate a motor or other drive system that automatically raises (or lowers) UV source 540 out of (or into) shield 516. Control system 570 may alternatively or further provide a wired or wireless communication interface, e.g., a Wi-Fi interface, that enables remote operation of UV disinfector 500. For example, a user of an app on a phone, a tablet, or other computing or communication system can communicate with control system 570 to control operation of UV disinfector 500. A user may thus be able to remotely schedule operation of multifunction disinfector 500 to automatically switch from air disinfection to surface disinfection at a specific time when the environment of UV disinfector 500 is expected to be unoccupied, e.g., at night, and automatically switch back to air disinfection when surface disinfection is complete or when the environment is expected to be occupied. Control system 570 can then direct the shield system of UV disinfector 500 to automatically raise or lower UV source 540 at the scheduled times and initiate surface or air disinfection without requiring that a person be present in the environment to manually change the state of UV disinfector.

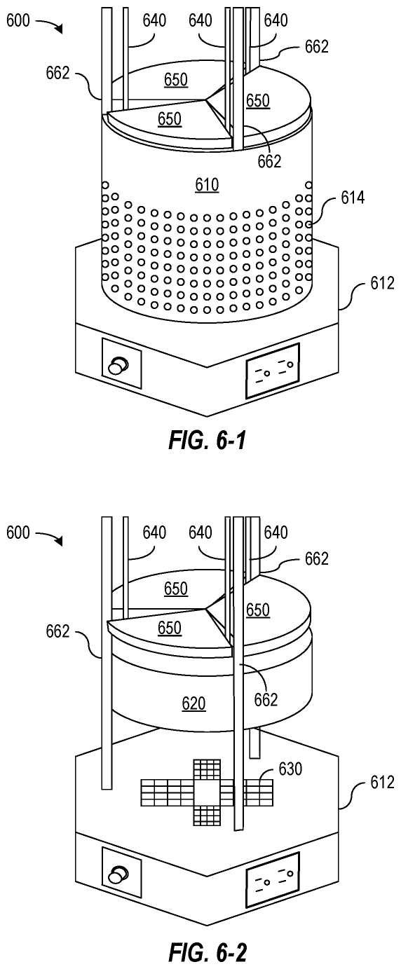

FIGS. 6-1 and 6-2 illustrate an arrangement of functional systems in the base 612 of a multifunction UV disinfector 600 in accordance with an example of the present disclosure. Disinfector 600 particularly includes a needle point bipolar ionizer (NPBI) 630 on base 612. A blower 620 is positioned on a framework or slides 662 above NPBI 630 and operates during air disinfection to draw an air flow from the surrounding environment through a vent 614 for in a housing portion 610. The air flow from blower 620 passes through a HEPA filter 650 into a volume extending along UV sources 640. Another portion (not shown) of the housing for UV disinfector 600 guides the air flow past UV sources 640 to an exhaust vent (not shown) for UV disinfector 600.

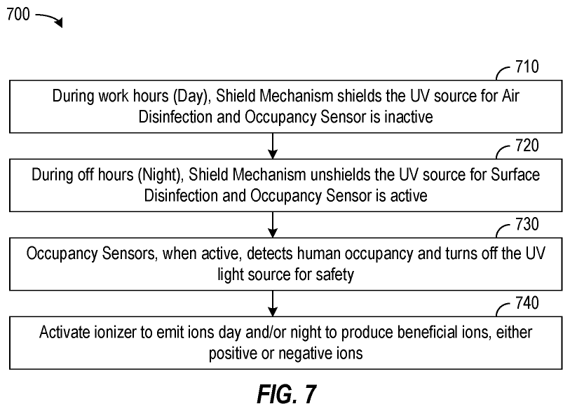

FIG. 7 is a flow diagram a process 700 for operating a multifunction UV disinfector such as described above. A process block 710 represents operation during work hours, e.g., during the day or other times when a room or other environment containing the UV disinfector is expected to have occupants. During process block 710, the housing and shield mechanism of the UV disinfector shields the UV source, e.g., keeps covers or shields closed, for air disinfection. The closing of the shield mechanism may either be a manual process or may be automated using motors in the shield mechanism. The occupancy sensor of the UV disinfector may be inactivated or disregarded during process block 710 to use UV disinfection of air while the environment is occupied.

A process block 720 represents operation during off hour, e.g., at night or when the environment is expected to be unoccupied. During process block 720, the shield mechanism opens, e.g., raises the covers or shields, to expose the UV sources in the UV disinfector for surface disinfection. The occupancy sensor for the UV disinfector is active during process block 720, i.e., during off hours, and a process block 730 shows the operation of the occupancy sensor, when active, shuts off the UV light source in the UV disinfector for the safety of any occupants in the environment around the UV disinfector.

An ionizer in the UV disinfector may be active during work and/or off hours to produce beneficial positive or negative ions. The ions may be beneficial in improving the efficiency of filters trapping particles that otherwise would be too small to filter out. Ionizers are helpful if users have allergies, asthma, or chemical sensitivities, as ionizers effectively remove pollutants ranging from pollen, mold, dust, and pet dander to viruses, smoke, odors, and chemical toxins.

Although particular implementations have been disclosed, these implementations are only examples and should not be taken as limitations. Various adaptations and combinations of features of the implementations disclosed are within the scope of the following claims.

What is claimed is:

1. A disinfector comprising:
a blower;
a UV source; and
a housing containing the UV source, the housing including a shield mechanism providing a closed configuration that directs an air flow created by the blower past the UV source for air disinfection and shields a surroundings of the disinfector from UV light from the UV source, the shield mechanism further providing an open configuration in which the UV light from the UV source at disinfecting intensity is directed to surfaces in the surroundings of the disinfector.

2. The disinfector of claim 1, further comprising a filter positioned to trap particles from the air flow created by the blower.

3. The disinfector of claim 2, wherein the UV source is positioned to irradiate the filter.

4. The disinfector of claim 2, wherein the filter comprises a HEPA filter.

5. The disinfector of claim 1, further comprising an ionizer positioned in the air flow created by the blower.

6. The disinfector of claim 5, wherein the ionizer comprises a needle point bipolar ionizer.

7. The disinfector of claim 1, further comprising an occupancy detector connected to the UV source, the occupancy detector turning off the UV source when the shield mechanism is in the open configuration and the occupancy detector senses an occupant around the UV disinfector.

8. The disinfector of claim 1, wherein the shield system comprises a panel that in the closed configuration is in an opening through the housing and blocks the UV light from escaping through the opening, the panel being removed from the opening in the open configuration.

9. The disinfector of claim 1, wherein the shield system comprises a cover that surrounds the UV source in the closed configuration.

10. The disinfector of claim 9, wherein the cover is removed from disinfector in the open configuration.

11. The disinfector of claim 9, wherein the cover has a sliding mount and is slid away from the UV source in the open configuration.

12. The disinfector of claim 9, wherein the UV source has a sliding mount and is slid away from the cover in the open configuration.

13. The disinfector of claim 1, wherein the shield system comprises motorized drive connected to shift the shield system between the closed configuration and the open configuration.

14. The disinfector of claim 1, further comprising a control system with a communication interface providing remote control and scheduling of operations of the disinfector.

15. The disinfector of claim 1, further comprising:
a filter positioned to trap particles from the air flow created by the blower; and
an ionizer positioned in the air flow created by the blower.

16. The disinfector of claim 15, further comprising an occupancy detector connected to the UV source, the occupancy detector turning off the UV source when the shield mechanism is in the open configuration and the occupancy detector senses an occupant around the UV disinfector.

17. The disinfector of claim 15, wherein the shield system comprises a panel that in the closed configuration blocks the UV light from escaping the disinfector, the panel being removed from the opening in the open configuration.

18. The disinfector of claim 15, wherein the shield system comprises a cover that surrounds the UV source in the closed configuration.

19. The disinfector of claim 18, wherein the cover is removed from disinfector in the open configuration.

20. The disinfector of claim 18, wherein the cover has a sliding mount and is slid away from the UV source in the open configuration.

21. The disinfector of claim 18, wherein the UV source has a sliding mount and is slid away from the cover in the open configuration.

22. A process for operating a UV disinfector, comprising:
placing the UV disinfector in an environment;
operating the UV disinfector in a closed configuration to circulate an air flow from the environment past a UV source inside the UV disinfector while UV light from the UV source inactivates contagions in the air flow, the closed configuration of the UV disinfector preventing the UV light from escaping the UV disinfector; and
operating the UV disinfector in an open configuration to irradiate a surface in the environment outside the UV disinfector with the UV light from the UV source and thereby inactivate contagions on the surface.

23. The process of claim 22, further comprising remotely controlling the disinfector to activate a drive system that shifts the disinfector between the closed configuration and the open configuration.

24. A disinfector comprising:
a blower;
a UV source; and
a housing containing the blower and the UV source and including a shield mechanism with a drive system connected to shift the disinfector between a closed configuration and an open configuration, wherein:
the closed configuration directs an air flow created by the blower past the UV source for air disinfection and shields an environment around the disinfector from UV light from the UV source: and
the open configuration passes the UV light from the UV source at disinfecting intensity to surfaces in the environment around the disinfector.

25. The disinfector of claim 24, further comprising a manually operated control that when operated activates the drive system to shift the disinfector between the closed configuration and the open configuration.

26. The disinfector of claim 24, further comprising a wireless communication interface providing remote control of the drive system.

27. The disinfector of claim 24, further comprising a control system configured to schedule of operation of the disinfector in the closed configuration for air disinfection and in the open configuration for surface disinfection.
